(19) United States
(12) Patent Application Publication
(10) Pub. No.: US 2023/0166051 A1
(43) Pub. Date: Jun. 1, 2023

(54) APPARATUS FOR APPLYING AN ENDOTHERMIC VAPOR TO SKIN AS AN ANESTHETIC

(71) Applicant: Vapocoolshot, Inc., Boca Raton, FL (US)

(72) Inventor: Jacob Leibovici, Wellington, FL (US)

(21) Appl. No.: 17/537,424

(22) Filed: Nov. 29, 2021

(51) Int. Cl. A61M 5/42

(52) U.S. Cl. CPC A61M 5/422

(57) ABSTRACT

The present device provides a method and an apparatus for applying a vaporized endothermic gas to a skin surface to provide numbing of the application site for injection with a syringe. The device includes a container or canister containing an endothermic gas that rapidly absorbs heat when released to the atmosphere. A depressible actuating member or trigger propels the gas or vapor through an outlet nozzle that is constructed to break the liquid into a vapor that can be projected as a stream of vapor along a delivery axis that intersects a delivery axis of the syringe needle; therefore, the gas or vapor can be successively delivered to an injection site with minimal repositioning of the device.

### PRIORITY CLAIM

The present invention is related to U.S. Pat. Application No. 16/017,379, entitled “METHOD AND APPARATUS FOR APPLYING AN ANESTHETIC AND BACTERICIDE”, filed Jun. 25, 2018, now U.S. Pat. No. 10,675,417, issued Jun. 9, 2020, which claims priority as a continuation-in-part of U.S. Pat. Application No. 15/094,754, entitled “METHOD AND APPARATUS FOR APPLYING AN ANESTHETIC AND BACTERICIDE”, filed Apr. 8, 2016, now U.S. Pat. No. 10,004,855, issued Jun. 26, 2018, which claims priority to U.S. Provisional Pat. Application No. 62/145,322, entitled “METHOD AND APPARATUS FOR APPLYING AN ANESTHETIC AND BACTERICIDE”, filed Apr. 9, 2015, which also claims priority as a continuation-in-part of U.S. Pat. Application No. 14/453,475, entitled “METHOD AND APPARATUS FOR APPLYING AN ANESTHETIC AND BACTERICIDE”, filed Aug. 6, 2014, now U.S. Pat. No. 9,656,028, issued May 23, 2017, which is a continuation-in-part of 13,927,454, entitled “METHOD AND APPARATUS FOR APPLYING AN ANESTHETIC AND BACTERICIDE”, filed Jun. 26, 2013, now U.S. Pat. No. 9,561,334, issued Feb. 7, 2017, which is a continuation-in-part of U.S. Pat. Application No. 12/557,753, entitled “METHOD AND APPARATUS FOR APPLYING AN ANESTHETIC”, filed Sep. 11, 2009, now U.S. Pat. No. 8,500,678, issued Aug. 6, 2013, which is a continuation-in-part of U.S. Application No. 11/636,859, entitled “DENTAL SYRINGE”, filed on Dec. 11, 2006, now abandoned, which claims the priority to U.S. Provisional Pat. Application No. 60/733,757, entitled “CRYO-SYRINGE”, filed on Mar. 7, 2006. The contents of which the above referenced applications are incorporated herein by reference.

### FIELD OF THE INVENTION

Generally, the invention relates to an apparatus for applying an endothermic gas to tissue to provide an anesthetic to an injection site prior to an injection to minimize the pain associated with conventional injection techniques. In particular, the apparatus comprises a receptacle for a liquefied endothermic gas and system for converting the liquefied endothermic gas to a vapor for application to the skin.

### BACKGROUND

Syringes are employed millions of times daily all over the world to inject medicines into people, as well as animals. Many times, injections are made in areas of the body that are somewhat less sensitive to pain. Other locations of the body where injections are contemplated are significantly more sensitive to pain, and the patient feels a pinching sensation that may be quite painful as the syringe needle is inserted beneath the skin. Such areas include, for example, gums and areas of the face, such as the forehead, as well as the lips. To minimize the pain that results when the injection needle penetrates, for example, a patient’s gums, the dental practitioner will often apply a topical agent to the injection site using a cotton swab. Because the deadening agent is only applied topically, it is not effective, as it does not cross the skin/mucosal membranes and misleads the patient into a false expectation of a painless injection. As a result, injecting an anesthetic often causes significant pain at the injection site. In other cases, such as diabetics, patients may be required to self-medicate on a daily basis. The repeated injections often create sensitive areas where injections are painful to the patient. This pain may cause patients to delay or omit medication to avoid the pain associated therewith. Current devices, even those to the present inventor, that utilize cold sprays to numb the injection site suffer from applying the numbing agent as a liquid pressurized spray. The liquid spray fails to optimally atomize for proper cooling of the skin. The vaporization of the liquid is slowed, and thus the cooling effect is reduced for a given amount of spray applied.

### SUMMARY

There is currently a need for a means of minimizing the pain associated with an injection. The present invention addresses this need by providing a syringe having a liquid compressed gas canister securely attached. The device includes an endothermic gas compressed to a liquid form. When the liquid endothermic material is released, it is converted to a vapor that can be directionally guided to the injection point to rapidly absorb heat when released to the atmosphere. The endothermic vapor is applied to the injection site prior to an injection to minimize the pain associated with conventional injection techniques. Furthermore, the vapor also blanches the mucosa almost instantly, allowing a practitioner to readily identify the pretreated injection site so that the needle is not inserted into an unanesthetized area.

Embodiments of the invention are also directed to an apparatus comprising a syringe which is removably attached to a cold spray module for converting the liquefied gas to a vapor by impingement. The cold spray module is secured to a compressed gas assembly which holds the liquefied endothermic gas. The apparatus comprises an actuating member which acts to dispense the contents of a container or canister containing the anesthetic composition. The cold spray module includes an actuating member for the controlled release of the liquefied endothermic gas for conversion to a vapor.

Embodiments of the invention are further directed to a liquefied endothermic gas canister assembly for attachment to the present device. The canister assembly includes a dispensing valve that is secured to a locking collar. The combination of the dispensing valve and the locking collar prevent other types of pressurized canisters from being secured to the dispensing valve for the present device, thereby reducing or preventing the present device from being used with unknown gaseous materials. The combination also prevents the gas cylinders from being refilled with the wrong compressed gas.

Other aspects are described infra.

### BRIEF DESCRIPTION OF THE DRAWINGS

FIG. 1 is a perspective view of the cold spray module with the gas cylinder and syringe installed and ready for use;

FIG. 2 is a perspective view of the cold spray module with the gas cylinder assembly and the syringe detached;

FIG. 3 is a partially exploded view illustrating the gas cylinder assembly with storage cap and cold spray module ready for connection;

FIG. 4 is a perspective view of one embodiment of the cold spray module connected to the gas cylinder assembly;

FIG. 5 is a perspective view of the embodiment illustrated in FIG. 4;

FIG. 6A is a perspective view illustrating connection of the cold spray module to a gas release valve securable to the gas cylinder;

FIG. 6B is a perspective view illustrating connection of the cold spray module to a gas release valve securable to the gas cylinder;

FIG. 6C is a perspective view illustrating connection of the cold spray module to a gas release valve securable to the gas cylinder;

FIG. 6D is a perspective view illustrating connection of the cold spray module to a gas release valve securable to the gas cylinder;

FIG. 6E is a perspective view illustrating connection of the cold spray module to a gas release valve securable to the gas cylinder;

FIG. 7A is a front view of the gas cylinder assembly;

FIG. 7B is a side view of the gas cylinder assembly of FIG. 7A;

FIG. 7C is a section view taken along lines 7C-7C of FIG. 7B;

FIG. 7D is an enlarged partial section view taken along lines 7D-7D of FIG. 7C illustrating the locking ring for providing a tamper resistant gas cylinder assembly;

FIG. 7E is a top view of the gas cylinder assembly of FIG. 7A;

FIG. 7F is a partially exploded view illustrating the lock ring of the gas cylinder assembly;

FIG. 7G is a partial perspective view of the gas cylinder assembly of FIG. 7A;

FIG. 8 is an exploded view of the gas cylinder assembly illustrated in FIGS. 7A-7G;

FIG. 9 is an alternative embodiment of the gas cylinder assembly constructed without the locking ring;

FIG. 10A is a top left perspective view of the preferred embodiment of the actuator for the gas cylinder assembly;

FIG. 10B is a bottom rear perspective view of the actuator of FIG. 10A;

FIG. 10C is a front view of the actuator shown in FIG. 10A;

FIG. 10D is a section view taken along lines 10D-10D of FIG. 10C;

FIG. 10E is a left side view of the actuator of FIG. 10A;

FIG. 10F is a section view taken along lines 10F-10F of FIG. 10E;

FIG. 10G is a bottom view of the actuator of FIG. 10A;

FIG. 10H is a section view taken along lines 10H-10H of FIG. 10G;

FIG. 10I is a rear view of the actuator of FIG. 10A;

FIG. 10J is a top view of the actuator of FIG. 10A;

FIG. 10K is a section view taken along lines 10K-10K of FIG. 10J;

FIG. 11A is a perspective view of a cover cap assembly for the gas cylinder assembly;

FIG. 11B is a front view of the cover cap assembly shown in FIG. 11A;

FIG. 11C is a right side view of the cover cap assembly shown in FIG. 11A;

FIG. 11D is a top view of the cover cap assembly shown in FIG. 11A;

FIG. 11E is a bottom view of the cover cap assembly shown in FIG. 11A;

FIG. 11F is a section view taken along lines 11F-11F of FIG. 11B;

FIG. 11G is a partial section view taken along lines 11G-11G of FIG. 11F;

FIG. 12A is a perspective top front view of one embodiment of the actuator of the present invention;

FIG. 12B is a top rear view of the actuator illustrated in FIG. 12A;

FIG. 12C is a front view of the actuator illustrated in FIG. 12A;

FIG. 12D is a right side view of the actuator illustrated in FIG. 12A;

FIG. 12E is a bottom view of the actuator illustrated in FIG. 12A;

FIG. 12F is a top view of the actuator illustrated in FIG. 12A;

FIG. 12G is a left side view of the actuator illustrated in FIG. 12A;

FIG. 12H is a section view taken along lines 12H-12H of FIG. 12C;

FIG. 12I is a front view taken along lines 12I-12I of FIG. 12D;

FIG. 12J is a partial view taken along lines 12J-12J of FIG. 12F;

FIG. 13A is a top perspective view of the locking ring;

FIG. 13B is a top view of the locking ring of FIG. 13A;

FIG. 13C is a section view taken along lines 13C-13C of FIG. 13B;

FIG. 14A is a partial perspective view illustrating one embodiment of the cold spray module;

FIG. 14B is an exploded view of the portions of the cold spray module of FIG. 14A;

FIG. 14C is a section view taken along the longitudinal centerline, illustrating the cold spray module secured to a gas cylinder assembly;

FIG. 15 is a section view of one embodiment of the cold spray assembly secured to a gas cylinder assembly;

FIG. 16 is an exploded view of the cold spray assembly of FIG. 15;

FIG. 17A is a perspective view of the cold spray assembly;

FIG. 17B is an exploded view of the cold spray assembly of FIG. 17A;

FIG. 18A is a front left perspective view of the cold spray housing;

FIG. 18B is a rear right perspective view of the cold spray housing;

FIG. 18C is a rear plan view of the cold spray housing;

FIG. 18D is a bottom view of the cold spray housing;

FIG. 18E is a right side view of the cold spray housing;

FIG. 18F is a front view of the cold spray housing;

FIG. 18G is a top view of the cold spray housing;

FIG. 18H is a partial section view taken along lines 18H-18H of FIG. 18C;

FIG. 18I is a section view taken along lines 18I-18I of FIG. 18D;

FIG. 18J is a partial section view taken along lines 18J-18J of FIG. 18I;

FIG. 19A is a front left perspective view of one embodiment of the cold spray housing;

FIG. 19B is a rear right perspective view of the cold spray housing of FIG. 19A;

FIG. 19C is a rear right perspective view of the cold spray housing of FIG. 19A;

FIG. 19D is a front view of the cold spray housing of FIG. 19A;

FIG. 19E is a bottom view of the cold spray housing of FIG. 19A;

FIG. 19F is a left side view of the cold spray housing of FIG. 19A;

FIG. 19G is a rear view of the cold spray housing of FIG. 19A;

FIG. 19H is a section view taken along lines 19H-19H of FIG. 19D;

FIG. 19I is a partial section view taken along lines 19I-19I of FIG. 19G;

FIG. 19J is a right side view of the cold spray housing of FIG. 19A;

FIG. 19K is a section view taken along lines 19K-19K of FIG. 19J;

FIG. 19L is a partial section view taken along lines 19L-19L of FIG. 19D;

FIG. 20A is a rear perspective view of a rear plate for the cold spray assembly;

FIG. 20B is a front perspective view of the rear plate of FIG. 20A;

FIG. 20C is a rear view of the rear plate of FIG. 20A;

FIG. 20D is a left side view of the rear plate of FIG. 20A;

FIG. 20E is a top view of the rear plate of FIG. 20A;

FIG. 20F is a bottom view of the rear plate of FIG. 20A;

FIG. 20G is a front view of the rear plate of FIG. 20A;

FIG. 20H is a section view taken along lines 20H-20H of FIG. 20C;

FIG. 20I is a section view taken along lines 20I-20I of FIG. 20G;

FIG. 21A is a front perspective view of one embodiment of the rear plate member;

FIG. 21B is a rear perspective view of the rear plate of FIG. 21A;

FIG. 21C is a front view of the rear plate of FIG. 21A;

FIG. 21D is a rear view of the rear plate of FIG. 21A;

FIG. 21E is a left side view of the rear plate of FIG. 21A;

FIG. 21F is a right side view of the rear plate of FIG. 21A;

FIG. 21G is a bottom view of the rear plate of FIG. 21A;

FIG. 21H is a top view of the rear plate of FIG. 21A;

FIG. 21I is a partial section view taken along lines 21I-21I of FIG. 21D;

FIG. 22A is a perspective view of a clamp block of the cold spray module;

FIG. 22B is a side view of the clamp block of FIG. 22A;

FIG. 22C is a rear view of the clamp block of FIG. 22A;

FIG. 22D is a side view of the clamp block of FIG. 22A;

FIG. 22E is a section view of the clamp block of FIG. 22A taken along lines 22E-22E of FIG. 22D;

FIG. 23A is a perspective view of a clamp block spring suitable for use with the present device;

FIG. 23B is a side view of the clamp block spring of FIG. 23A;

FIG. 23C is an end view of the clamp block spring of FIG. 23C;

FIG. 24A is a perspective view of one embodiment of the core tube of the cold spray module;

FIG. 24B is a side view of the core tube of FIG. 24A;

FIG. 24C is a front view of the core tube of FIG. 24A

FIG. 24D is a section view taken along lines 24D-24D of FIG. 24C;

FIG. 25A is a front perspective view of one embodiment of a core tube of the cold spray module;

FIG. 25B is a rear perspective view of the core tube of FIG. 25A;

FIG. 25C is a top view of the core tube of FIG. 25A;

FIG. 25D is a side view of the core tube of FIG. 25A;

FIG. 25E is a front view of the core tube of FIG. 25A;

FIG. 25F is a rear view of the core tube of FIG. 25A;

FIG. 25G is a section view taken along lines 25G-25G of FIG. 25D;

FIG. 25H is a section view taken along lines 25H-25H of FIG. 25C;

FIG. 26A is a perspective view of the nozzle body for the cold spray module;

FIG. 26B is a side view of the nozzle body of FIG. 26A;

FIG. 26C is a front view of the nozzle body of FIG. 26A;

FIG. 26D is a section view taken along lines 26D-26D of FIG. 26C;

FIG. 27A is a front perspective view of the supply tube for the cold spray module;

FIG. 27B is a side view of the supply tube of FIG. 27A;

FIG. 27C is an end view of the supply tube of FIG. 27A;

FIG. 28A is a rear perspective view of a breakup nozzle for the cold spray module;

FIG. 28B is a rear view of the breakup nozzle of FIG. 28A;

FIG. 28C is a front view of the breakup nozzle of FIG. 28A;

FIG. 28D is a section view taken along lines 28D-28D of FIG. 28B;

FIG. 29A is a front perspective view of a breakup nozzle for the cold spray module;

FIG. 29B is a rear perspective view of the breakup nozzle of FIG. 29A;

FIG. 29C is a side view of the breakup nozzle of FIG. 29A;

FIG. 29D is a side view of the breakup nozzle of FIG. 29A;

FIG. 29E is a front view of the breakup nozzle of FIG. 29A;

FIG. 29F is a rear view of the breakup nozzle of FIG. 29A;

FIG. 29G is a section view of the breakup nozzle of FIG. 29A taken along lines 29G-29G of FIG. 29C;

FIG. 30A is a front right perspective view of a breakup nozzle suitable for use with the present invention;

FIG. 30B is a rear perspective view of the breakup nozzle of FIG. 30A;

FIG. 30C is a side view of the breakup nozzle of FIG. 30A;

FIG. 30D is a front view of the breakup nozzle of FIG. 30A;

FIG. 30E is a rear view of the breakup nozzle of FIG. 30A;

FIG. 30F is a section view of the breakup nozzle of FIG. 30A taken along lines 30F-30F of FIG. 30E;

FIG. 31 is a perspective view of an embodiment having a flange retention member for attachment of the syringe;

FIG. 32 is a perspective view of an embodiment having a flange retention member for attachment of the syringe;

FIG. 33A is a perspective view illustrating the flange retainer;

FIG. 33B is a perspective view illustrating the flange retainer;

FIG. 34 is a perspective view of the attachment of the cold spray module to the gas cylinder;

FIG. 35A is a perspective view of the cold spray module with flange retention;

FIG. 35B is an exploded perspective view of the embodiment illustrated in FIG. 35A;

FIG. 36 is a front perspective view of the embodiment illustrated in FIG. 35A; and

FIG. 37 is a front perspective view of the embodiment illustrated in FIG. 35A.

### DETAILED DESCRIPTION

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

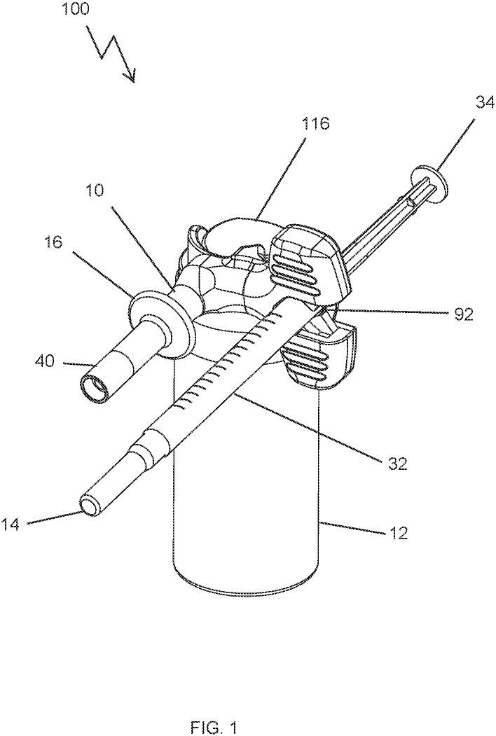

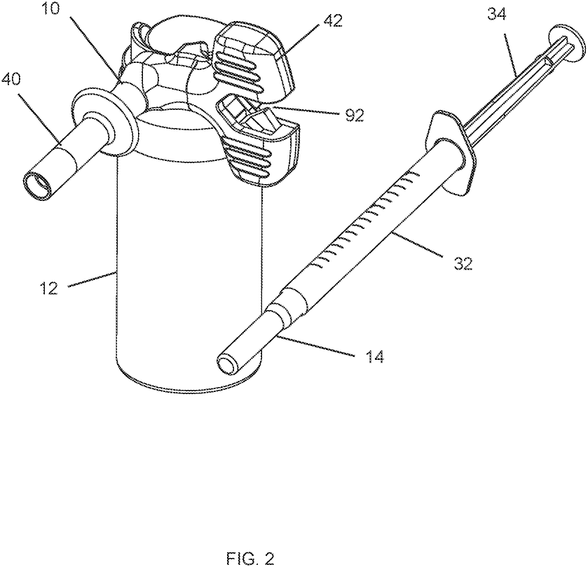

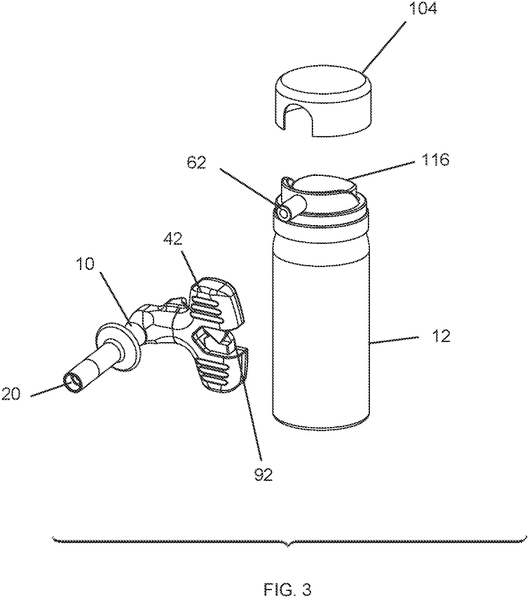

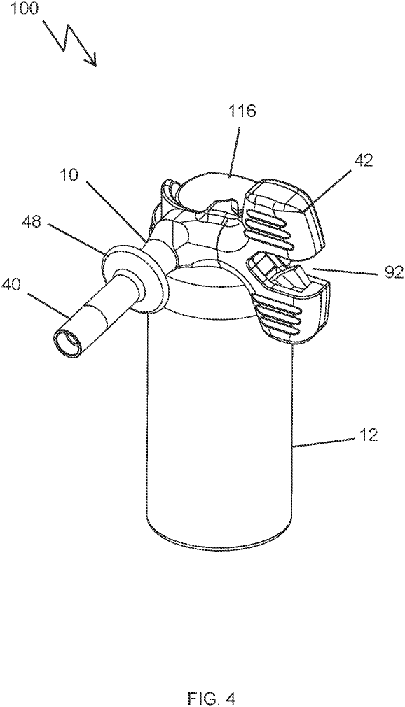

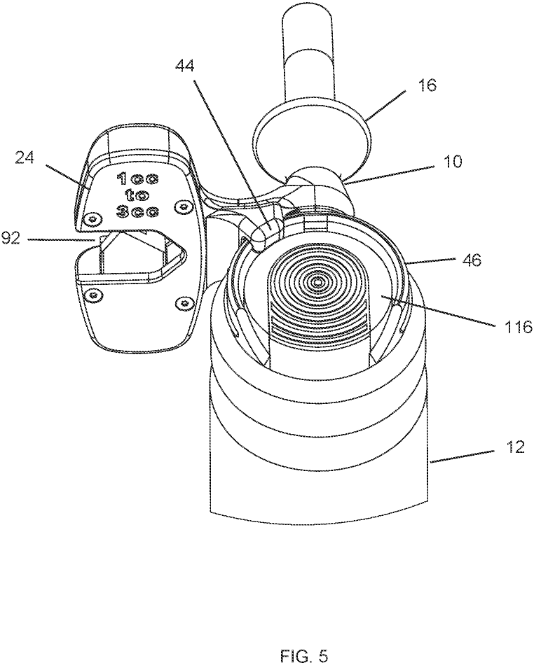

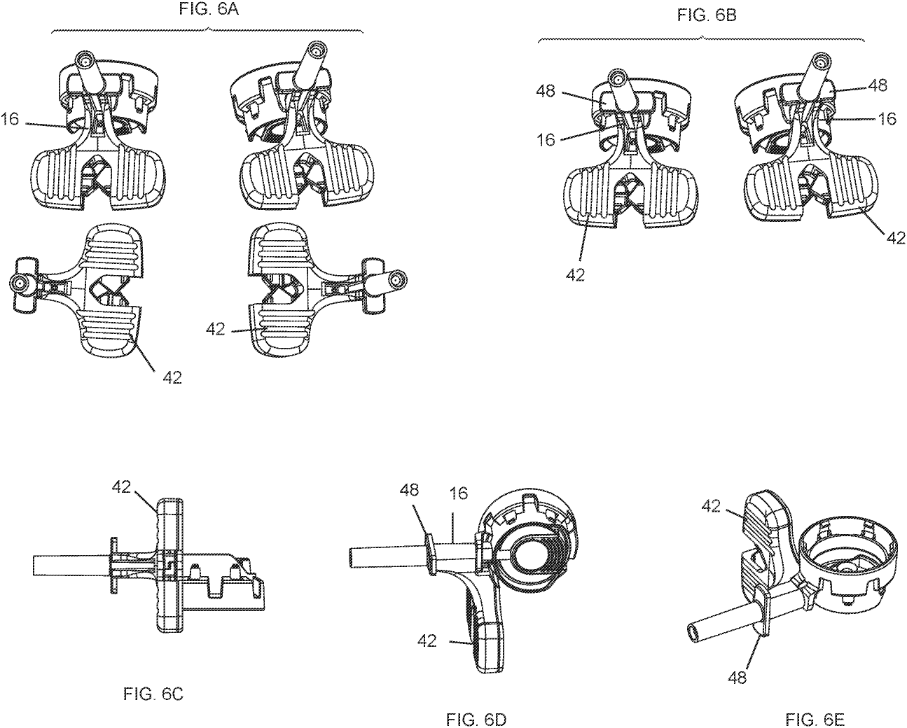

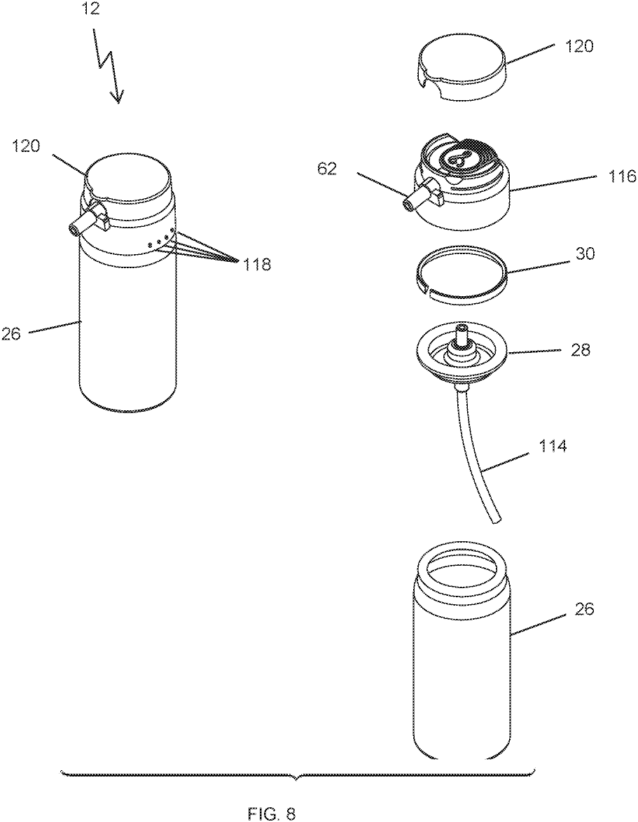

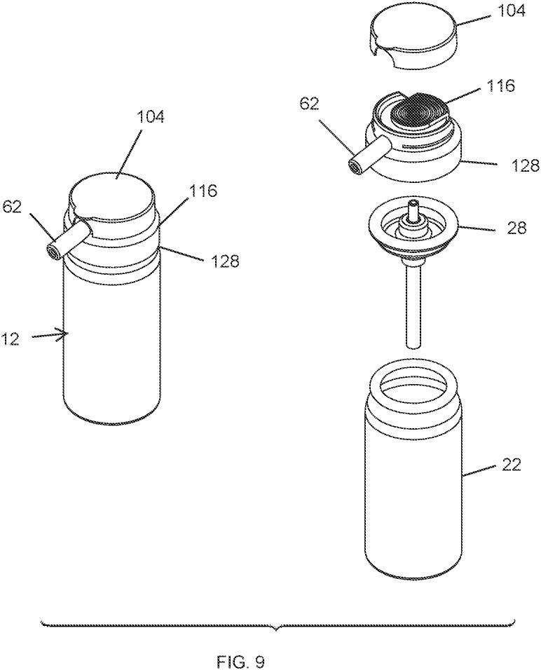

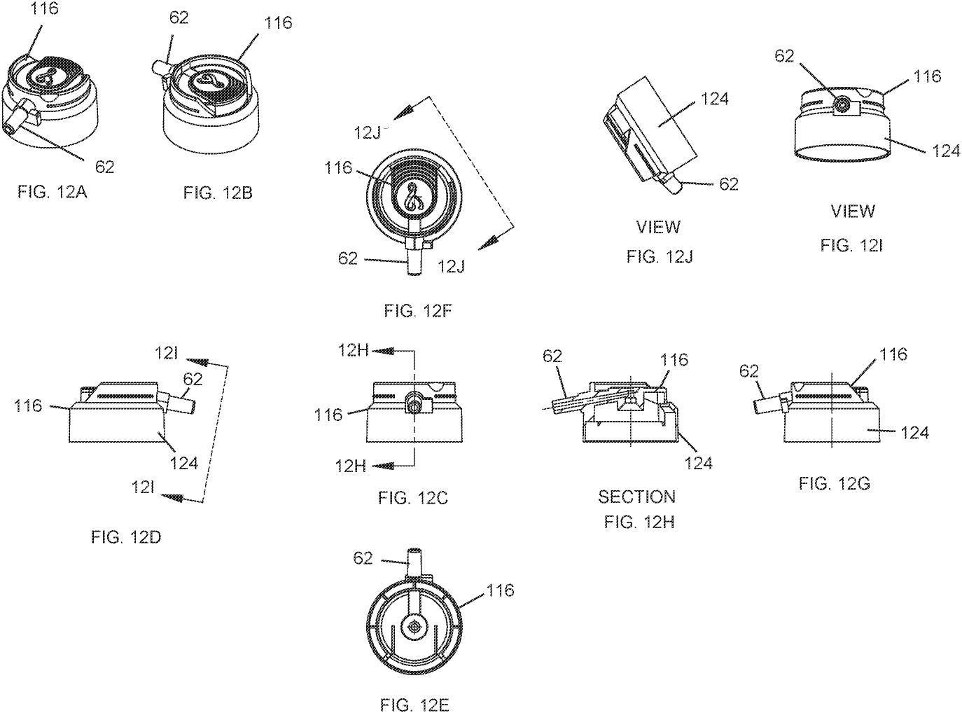

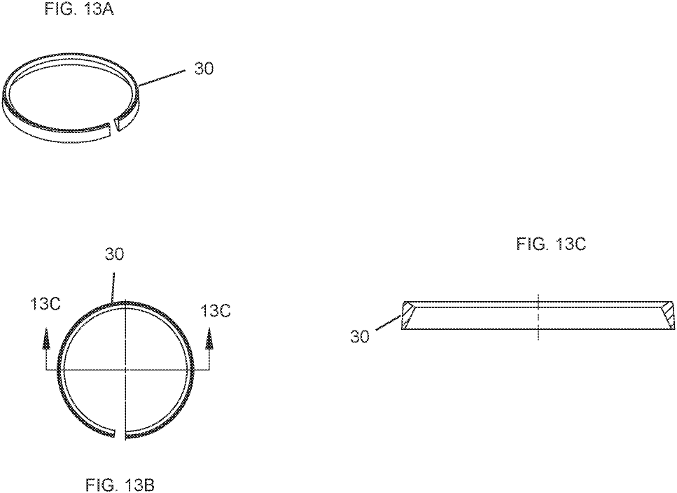

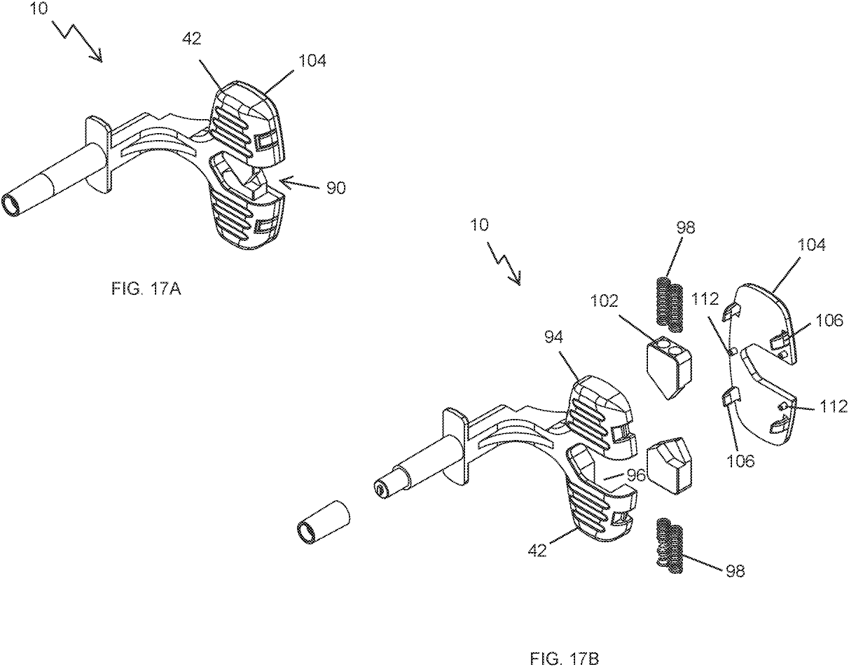

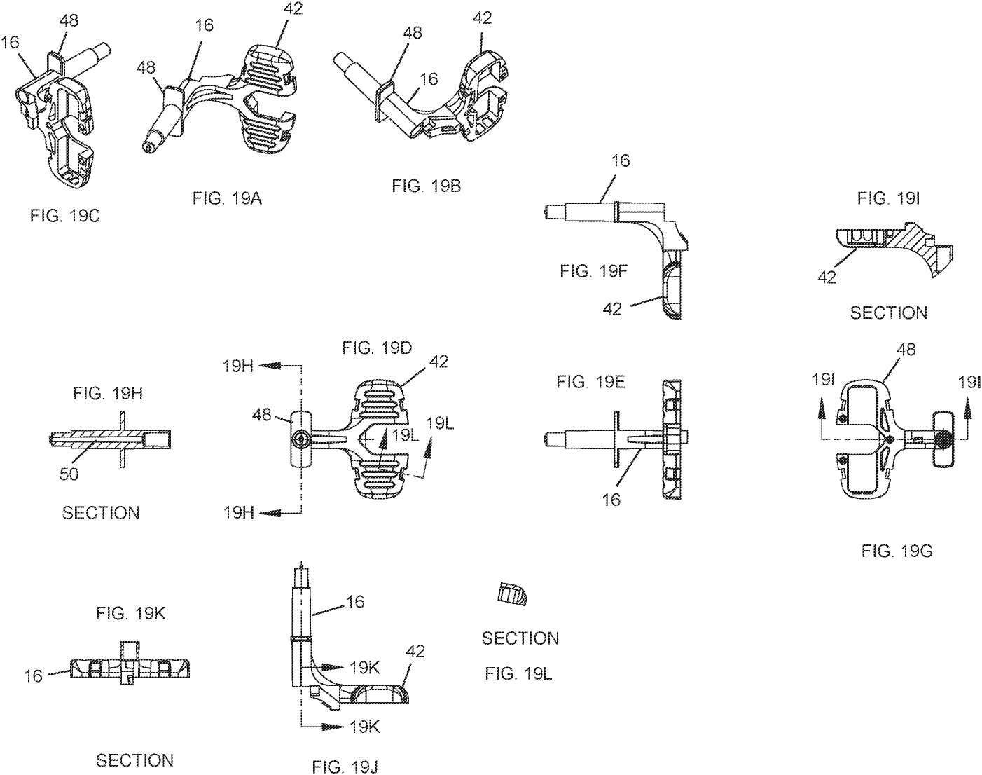

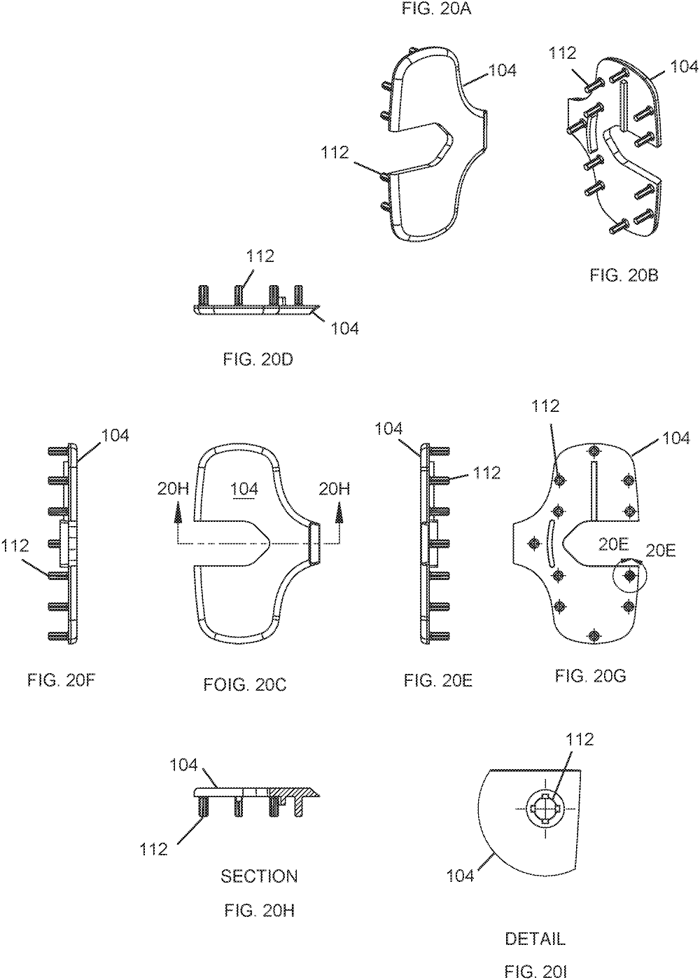

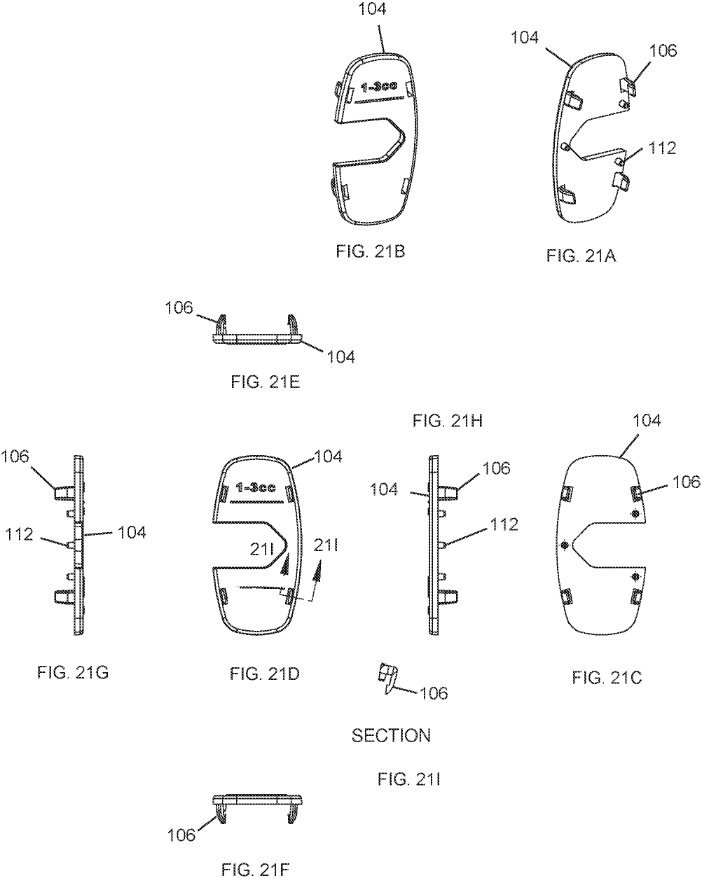

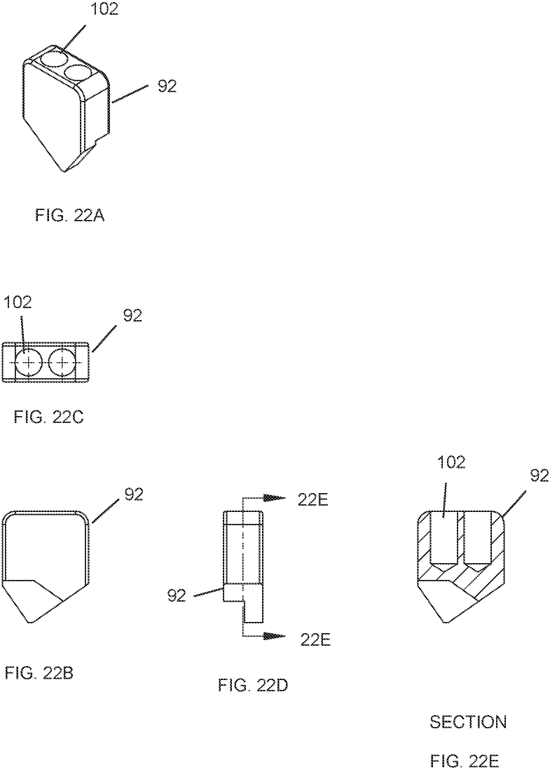

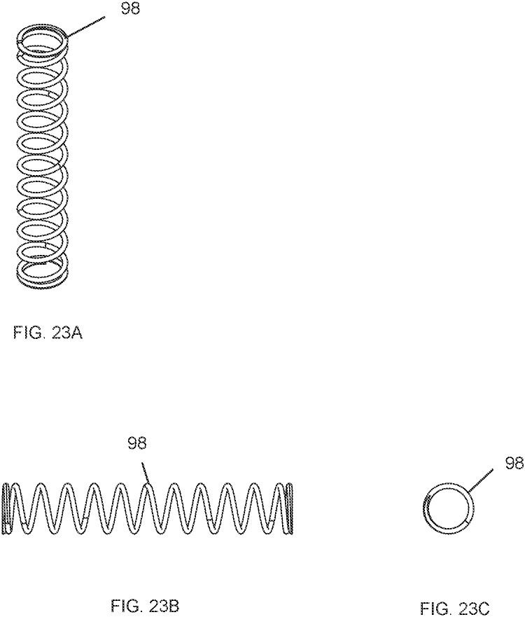

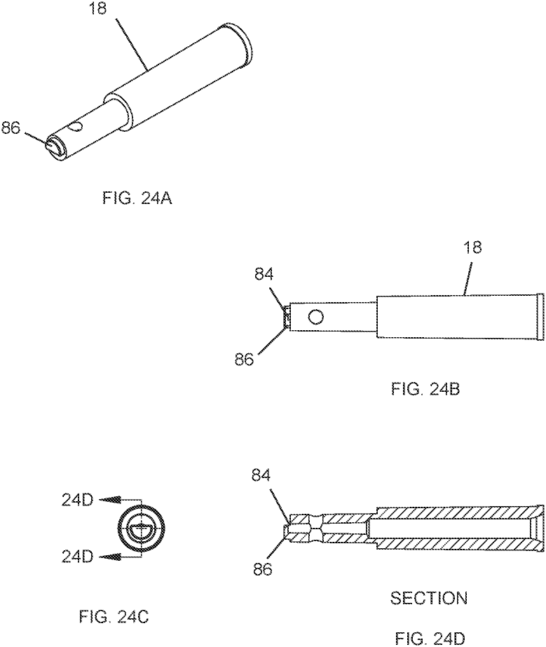

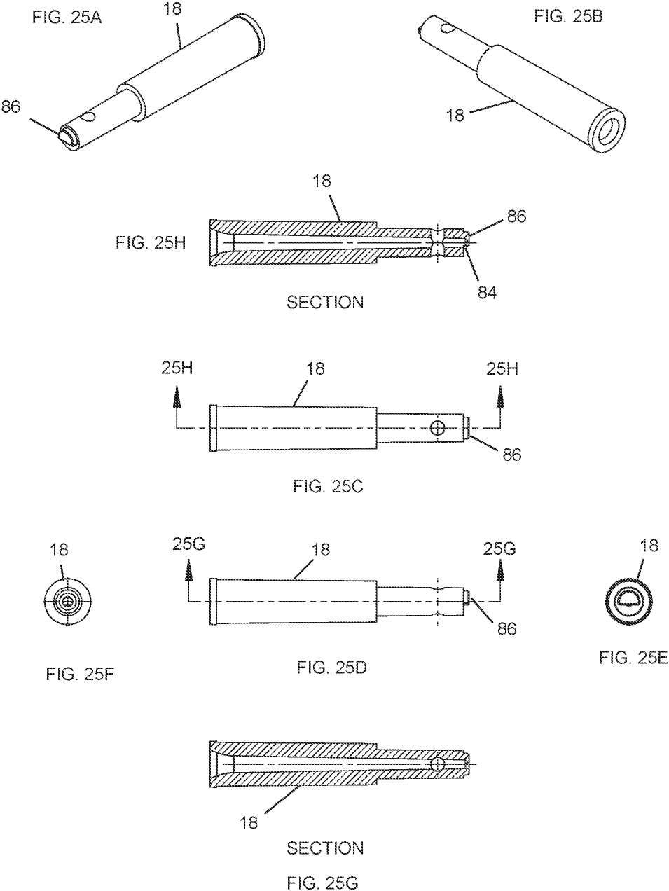

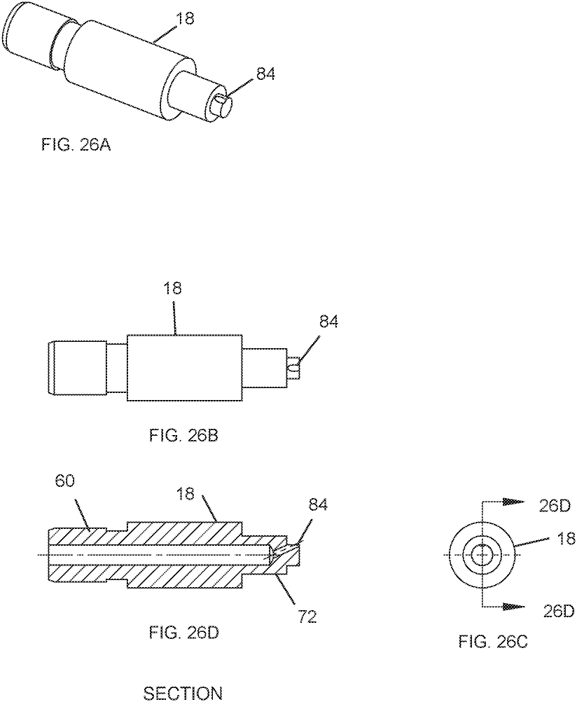

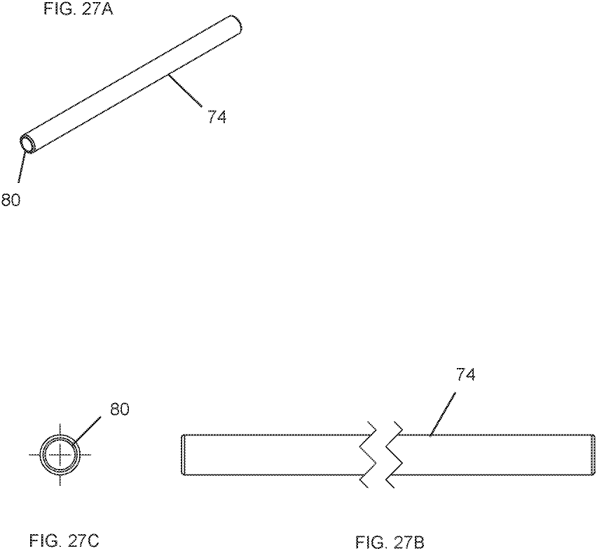

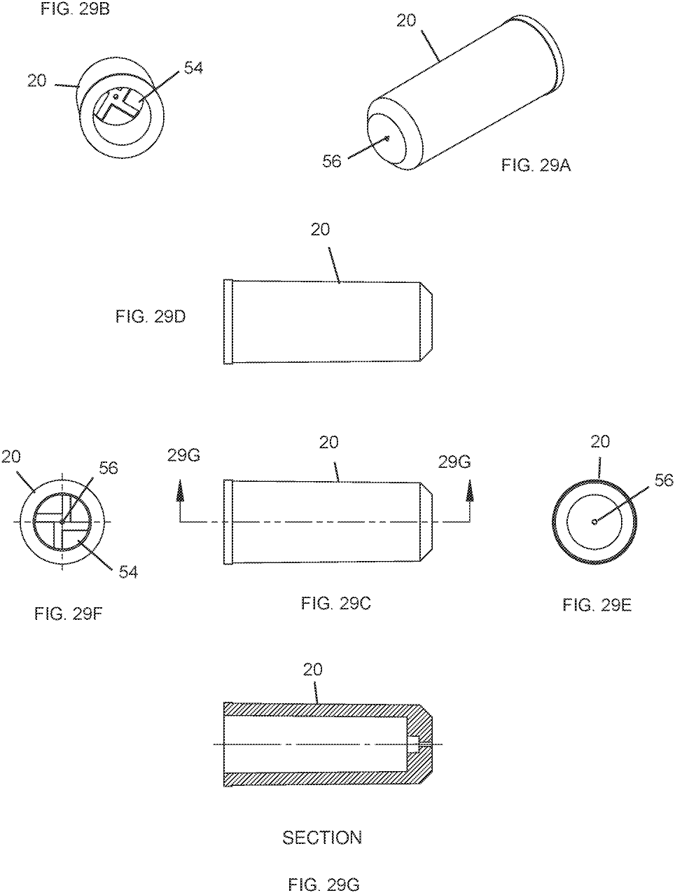

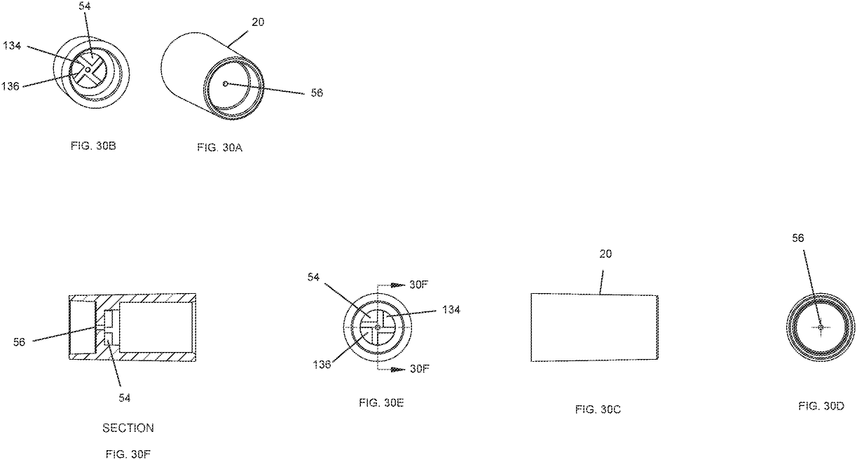

Referring to FIGS. 1-31, embodiments of the present invention relate to a method and apparatus of applying an anesthetic in the form of a vaporized endothermic gas. The endothermic vapor applicator 100 is attachable to a syringe to provide the endothermic vapor along a trajectory that aligns with the puncture point of the syringe needle. This construction allows the user to align the device once for the skin or epidermis, dermis numbing provided by the endothermic gas vapor as it cools the skin to be followed by the injection without the need for repositioning the device. The endothermic vapor applicator 100 includes a cold spray module 10, a canister assembly 12, and a syringe 14. The cold spray module 10 includes a cold spray module housing 16, a core tube 18, a breakup nozzle 20 and a syringe barrel securing assembly 90. The canister assembly 12 includes a canister 26, a valve assembly 28, and a locking ring 30. The syringe 14 includes a barrel 32, a plunger 34, a barrel tip 36 and a needle 38.

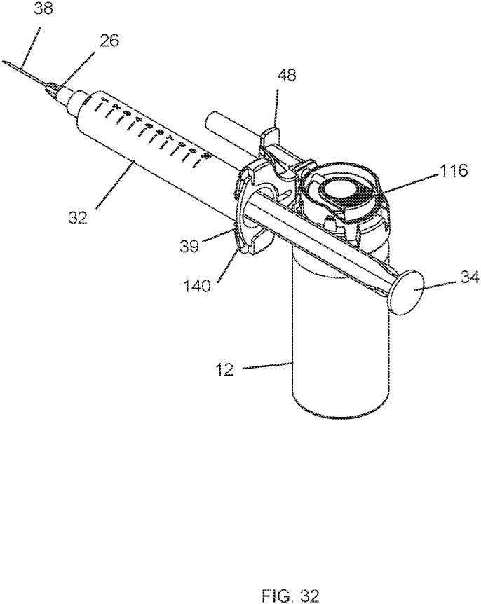

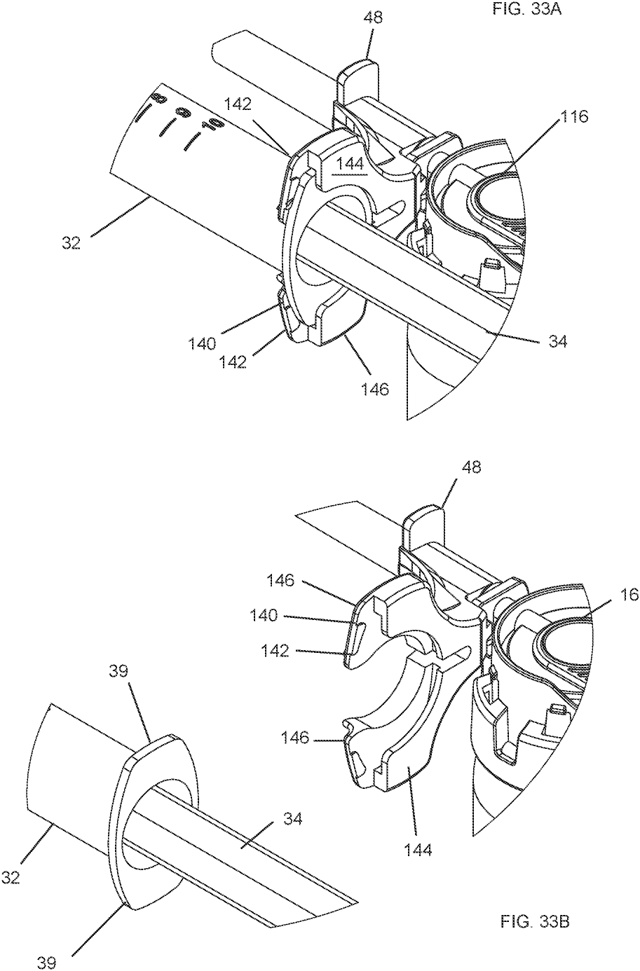

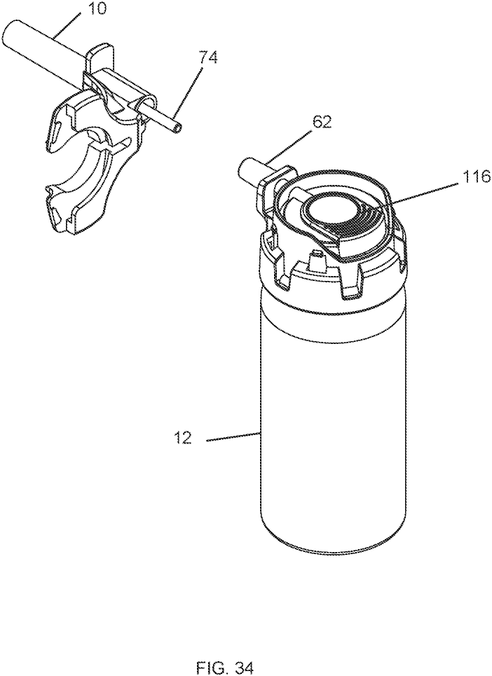

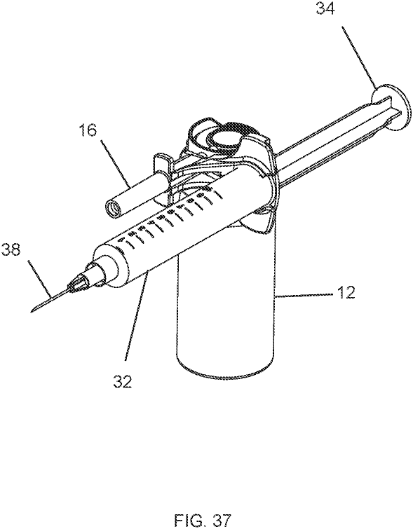

Referring generally to the Figures, and more specifically to FIGS. 1-5, 31-34 and 37, the endothermic vapor applicator 100 is illustrated as an assembly. In general, the cold spray module 10 is constructed and arranged for removable attachment to the canister assembly 12, as well as removably attached to the syringe 14 to connect the assemblies together in a manner that allows the cold vapor to be controllably sprayed and the syringe 14 to be positioned for operation of the cold spray and the syringe 14 with one hand. In this manner, the endothermic vapor applicator 100 can be aimed at a desired injection site and the endothermic gas released, for example, with the thumb. The thumb can then be repositioned to the plunger 34 of the syringe 14 for the injection. The endothermic vapor creates a blanched area on the skin, which is visible to insert the needle 38 of the syringe 14 into the numbed area. The ergonomic construction of the endothermic vapor applicator 100 allows both operations to occur without repositioning of the hand with respect to the cold spray module 10 and canister assembly 12 or visa-versa. Thus, the nozzle 20 is constructed and arranged to cause the endothermic vapor to be directed as a stream to intersect with a delivery axis of the needle 38 connected to the syringe 14 when the syringe 14 is attached to the cold spray module 100.

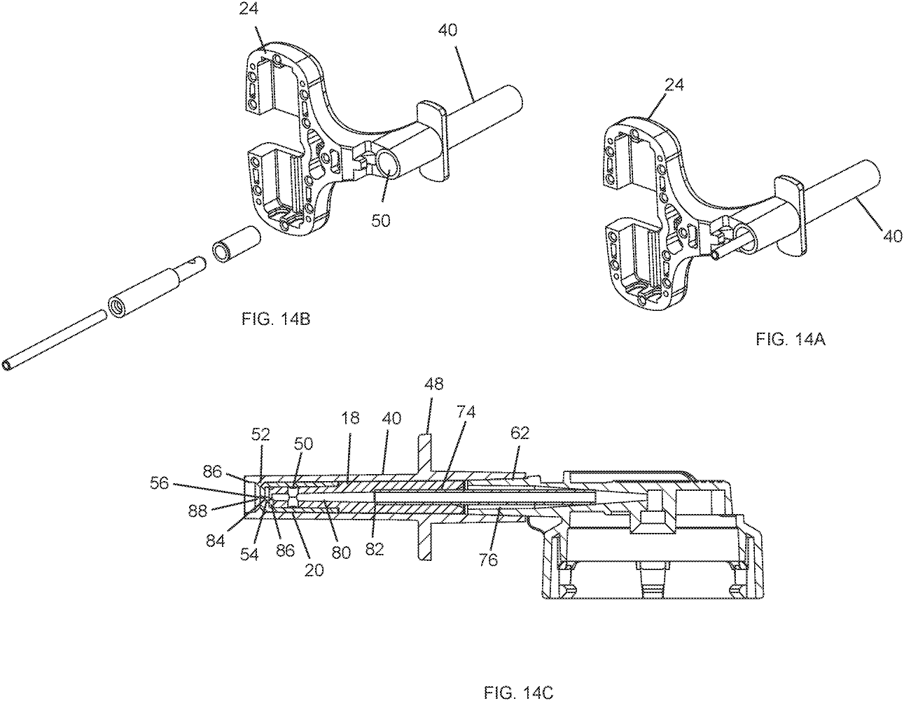

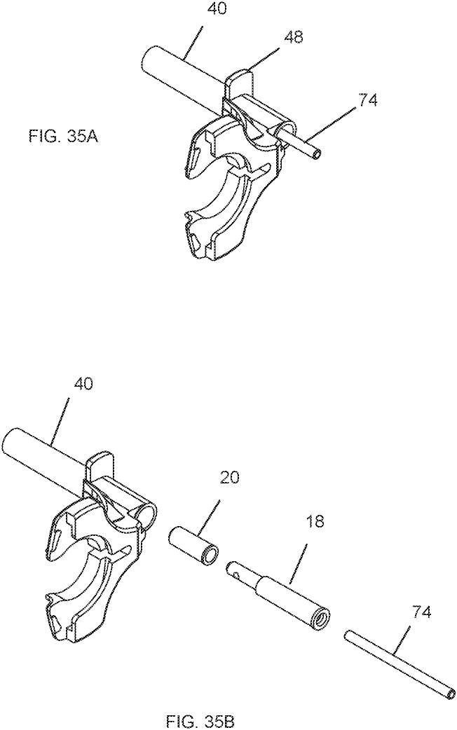

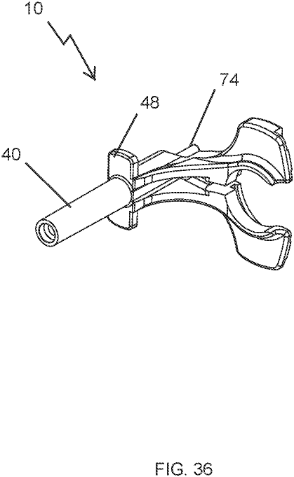

Referring generally to the Figures, and more specifically to FIGS. 1-6E, 14A-23C, and 31-37 , the cold spray module 10 is illustrated. The cold spray module 10 generally connects the syringe 14 and the canister assembly 12, and maintains the positioning of each assembly with respect to the other. In addition, the cold spray module 10 routes the liquefied endothermic gas from the canister assembly 12 to the desired area of the skin, breaking the liquid into a stream of vapor as it passes through the cold spray module 10. The cold spray module 10 includes a cold spray housing 16, a syringe barrel securing assembly 90, a core tube 18, and a breakup nozzle 20. The housing 16 is constructed generally as a tubular portion 40 having the syringe barrel securing assembly 90 depending therefrom, also forming a pair of finger grips 42. In some embodiments, a positioning anchor 44 extends from the area between the tubular portion 40 and the syringe barrel securing assembly 90 to snap onto the top rim 46 of the valve assembly 28 to prevent rotation and linear translation between the canister assembly 12 and the cold spray module 10. Secondary finger grips 48 may be integrally formed to the tubular portion 40 to provide versatility to gripping the endothermic vapor applicator 100. The internal bore 50 of the tubular portion 40 is generally a smooth, consistent or slightly tapered bore and may contain one or more internal flanges 52 for maintaining the position of the core tube 18 and breakup nozzle 20 within the bore. Thus, the core tube 18 seen in FIGS. 14A-16, and 24A-27C is inserted into the internal bore 50 of the tubular portion 40. The core tube 18 receives the liquefied endothermic gas from the canister assembly 12 and provides a pathway for the liquid to travel to a position where the liquid endothermic gas is directed to impinge at an angle upon a breakup surface 54 of the breakup nozzle 20 to convert the liquid to a vapor. The vapor is thus expanded to cause it to escape through the orifice under pressure to create a stream of endothermic vapor the user can direct at a skin surface.

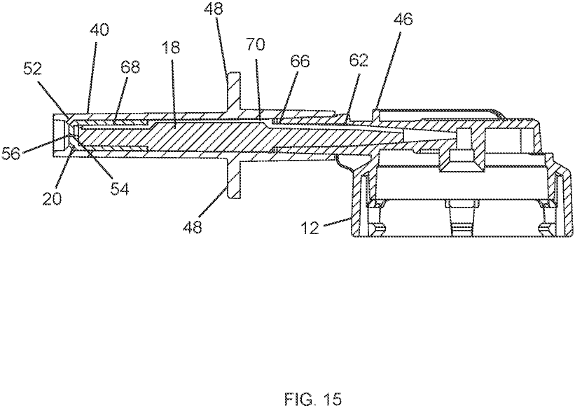

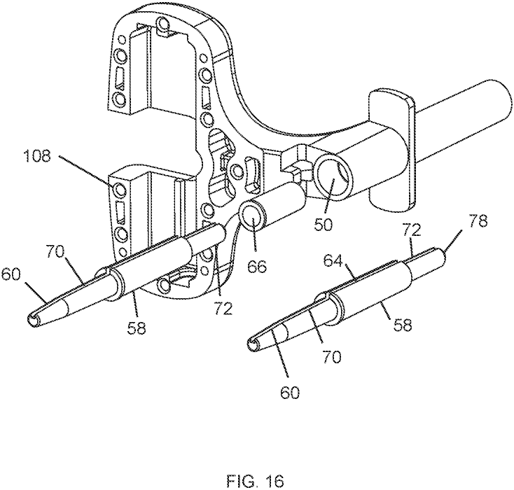

Referring generally to the Figures, and more specifically to FIGS. 14A-16, 24A-27C, and 35A-35B , various embodiments of the core tube 18 are illustrated. In general, the core tube 18 is constructed and arranged to be in fluid communication with the canister assembly 12 to transfer the compressed liquid endothermic gas from the canister 26 and direct the liquid into the breakup surface 54 of the breakup nozzle 20. Thus, the core tube 18 may be provided in various forms to provide the same or similar functions. FIGS. 15 and 16 illustrate an embodiment of the core tube 18 that includes an elongated body 58 sized to fit snugly into the tubular portion 40 of the cold spray module 10. A first end 60 of the core tube 18 is constructed to fit snugly into a discharge tube 62 of the canister valve 28, and a second end 72 is constructed to fit snugly into an inside bore 68 of the breakup nozzle 20. The snug fitment between the components allows the side surface 64 of the core tube 18 to include a U-shaped channel 70, whereby an internal surface 66 of the discharge tube 62, the internal bore 50 of the tubular portion 40 of the housing 16, and the inside bore 68 of the breakup nozzle 20 all form a closing surface for the U-shaped channel 70 to form a passage for the liquefied endothermic gas. It should be noted that the U-shaped channel 70 is positioned off center of the core tube 18, terminating in an annular groove 78 which directs the liquefied gas at a breakup surface 54 of the breakup nozzle 20. In this manner, the liquefied gas is required to impact the breakup surface 54 and travel across it at an angle to reach the orifice 56 of the nozzle 20. FIGS. 14A-14C, 26A-26D and 27A-27C illustrate an alternative embodiment of the core tube 18. In this embodiment, the first end 60 of the tube is a metal or polymeric tubing 74. An outer surface of the tubing 74 is constructed and arranged to cooperate with an inner surface 66 of the discharge tube 62. A centrally positioned conduit 80 extends from the distal end 82 of the tubing 74 to a discharge port 84. In a preferred embodiment, the discharge port 84 directs the liquefied gas against the breakup surface 54 of the nozzle 20 at an angle to break up the liquid into a vapor as it passes over the breakup surface 54. In at least one embodiment, the discharge port 84 is constructed by positioning a pair of offset flat panels 86 with a U-shaped notch 88 positioned in a central portion of the forward most flat panel. The offset positioning of the two panels 86 causes the liquid to exit the central conduit 80 at an angle with respect to the longitudinal centerline of the core tube 18. In some embodiments, see FIG. 14C, a tapered sleeve 76 may be overmolded or press-fit onto an outer surface of the tubing 74 to provide a snug fit to the inner surface 66 of the discharge tube 62 of the canister assembly 12. It should be noted that the term “snug” does not require a liquid or air tight seal, so long as the preponderance of the liquid is transferred through the core tube 18 to the breakup nozzle 20.

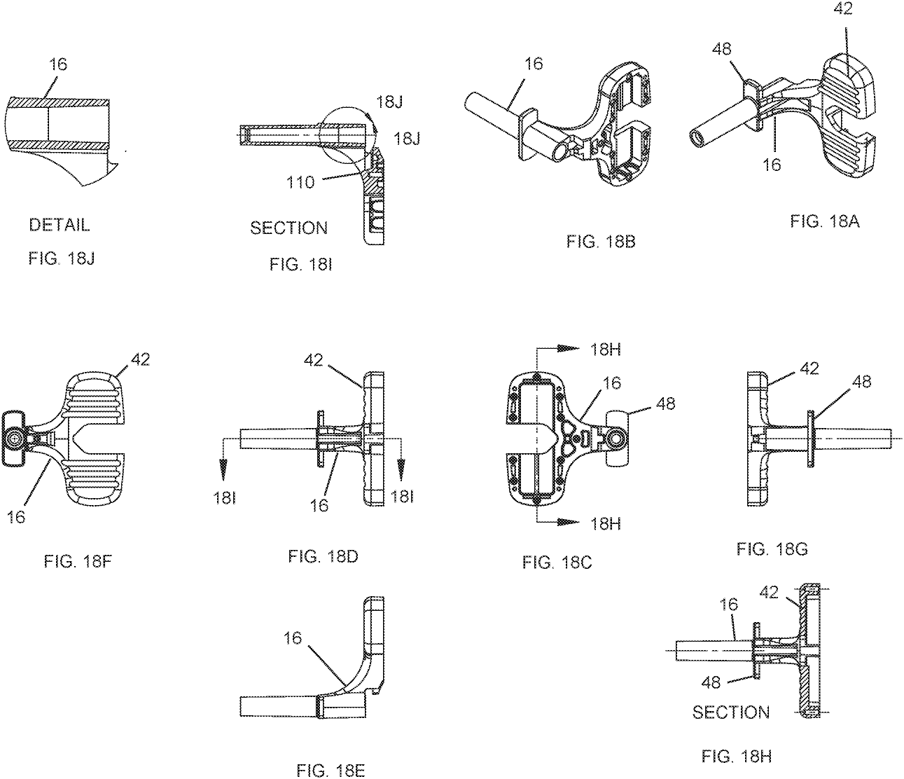

Referring generally to the Figures, and more specifically to FIGS. 1-6E, and 17A- 23C, wherein the cold spray module 10 includes a syringe barrel securing assembly 90; the syringe barrel securing assembly 90 including at least two opposing spring loaded clamping members 92 configured for securing the syringe barrel 14 to the cold spray module10. The cold spray module 10 is preferably configured to include a housing 94 having a first upper portion and a second lower portion formed to include channels or slots 96 sized and shaped to hold and allow the two opposing spring loaded clamping members 92 to slide therein. Springs 98 may be placed into pockets 102 positioned in the ends of the clamping members 92 to bias the clamping members to a closed or clamping position. While coil springs are illustrated, it should be noted that other biasing members, such as elastomeric, rubber or torsion members, may be utilized without departing from the scope of the invention. A cap member 104 is secured to the cold spray housing 94 with spring clips 106. The spring clips 106 are constructed and arranged to flex when inserted into blind apertures 108 (FIG. 16) as they slide past a catch member 110 (FIG. 18I) to interlock and prevent the removal of the cap member 104. Alignment pins 112 may be used in conjunction with the spring clips 106 to align the cap member 104 to the cold spray housing 94 to provide a more precise alignment between the two components. The alignment pins 112 may also be used without the spring clips 106 by providing a press fit into the blind apertures 108.

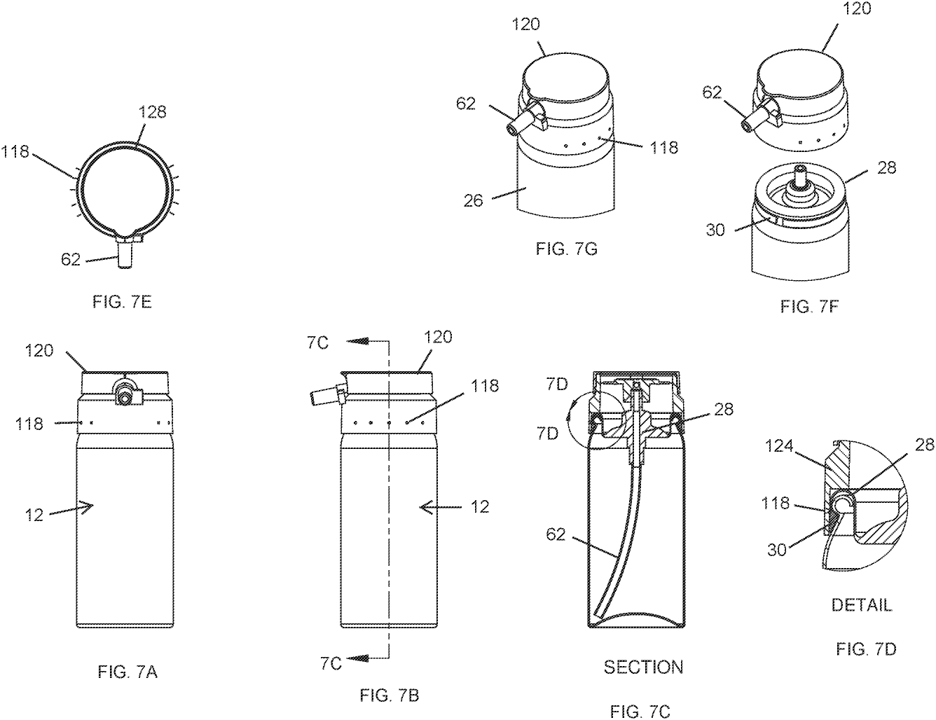

Referring generally to the Figures, and more specifically to FIGS. 7A-13C, embodiments of the canister assembly 12 are illustrated. The canister assembly 12 includes a metal canister 26 having a valve assembly 28 crimped and sealed to cover an open end of the canister to create a sealed pressure canister. In general, the canister assembly 12 is constructed and arranged to contain the liquefied endothermic gas sufficiently pressurized to maintain the liquid state of the gas until released from the canister 26. The valve assembly 28 includes a dispenser tube 114 extending outwardly therefrom for connection to the cold spray module 10 through a discharge tube 62 secured to a discharge actuator 116. The discharge actuator 116 is constructed and arranged to operate by depressing the dispenser tube 114 in the valve assembly 28, which allows the liquefied endothermic gas to flow through the valve assembly 28 and through the discharge tube 62. In at least one embodiment, a locking ring 30 is placed below the valve assembly crimp utilized to attach the valve assembly to the cannister. The locking ring 30 is laser or radio frequency welded 118 to the discharge actuator 116 to prevent removal of the discharge actuator 116 from the canister 26. This construction prevents unwanted refilling of the canister 26, which may allow contaminants or incorrect liquids into the system. Removal of the discharge actuator 116 requires destruction of the locking ring 30 as an obvious indicator of refilling or tampering of the canister 26. A cap 120 is constructed and arranged to cooperate with the discharge actuator 116 to prevent inadvertent operation thereof. Examples of endothermic liquefied gasses that can be used include, but should not be limited to, 1,1,1,3,3 Pentaflouropropane CAS—No.: 460-73-1 at greater than 90% of the mixture combined with 1,1,1,2 Tetraflouroethane CAS—No.: 811-97-2 making up the remaining portion. Alternatively, SOLSTICE® Propellant: HFO-1234ze(E) or SOLSTICE® Enhance: HFO-1233zd(E) mixed with 10% or less of PERIDEX™ chlorhexidine gluconate 0.12% may be utilized. The chlorhexidine gluconate is formed from 1,1¹ hexamethylene bis [5-(p-chlorophenyl) biguanide] di-D-gluconate) in a base containing water, 11.6% alcohol, glycerin, PEG-40 sorbitan diisostearate, flavor, sodium saccharin, and FD&C Blue No. 1. Peridex is a near-neutral solution (pH range 5-7). Chlorhexidine gluconate is a salt of chlorhexidine and gluconic acid. It should be noted that other combinations of liquefied endothermic gasses and materials, such as antibacterials, may be combined with the endothermic gasses to reduce the possibility of frostbite, while still reducing the temperature of the skin to the level necessary to reduce the pain of the injection.

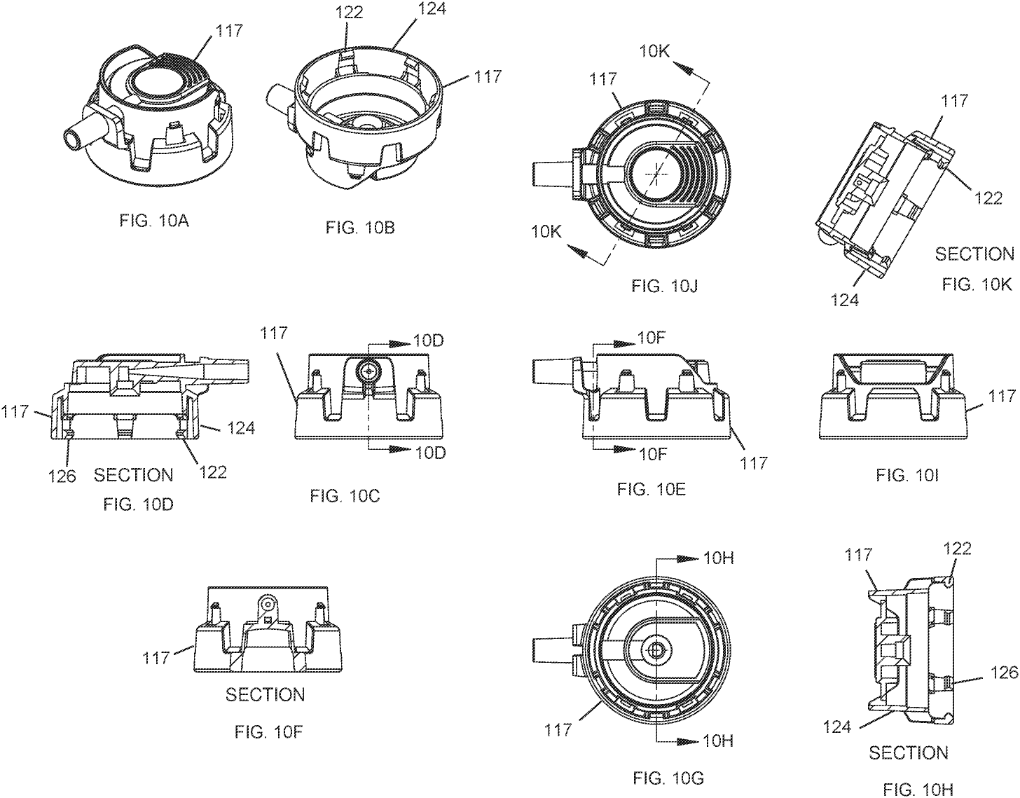

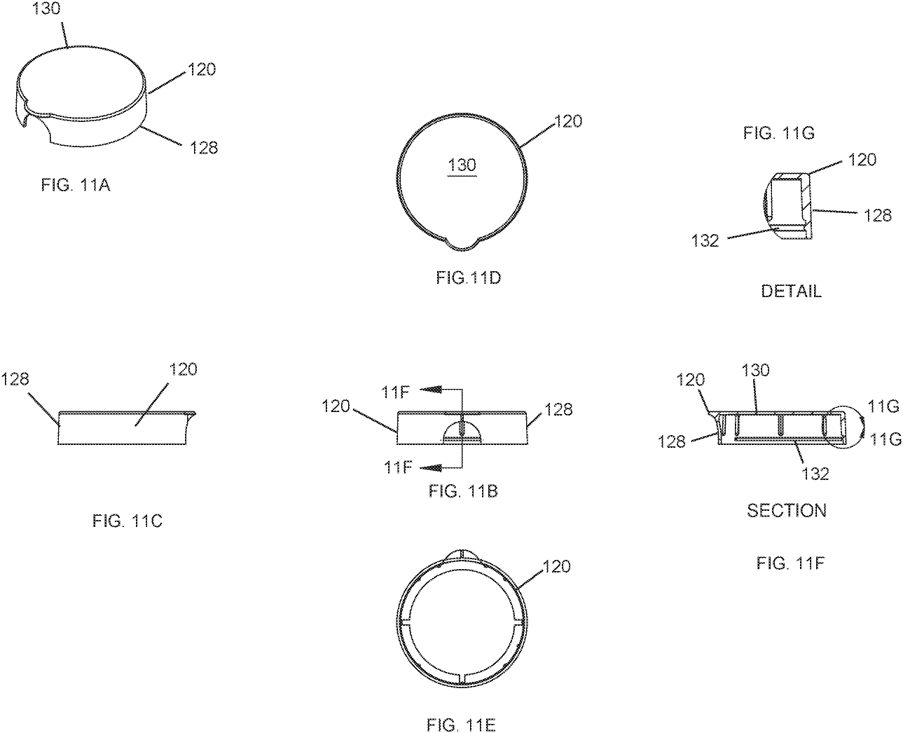

FIGS. 10A-10K illustrate an alternative embodiment of the discharge actuator 117. This embodiment is constructed to function the same as the discharge actuator labeled 116, with the exception of snap locks 122 formed into the skirt 124. The snap locks 122 snap over the crimped valve assembly 28 to hold the discharge actuator 117 in position; the snap locks 122 include a ramping surface 126 that allows the skirt 124 to expand as it passes over the valve assembly 28. Removal of the discharge actuator 117 from the crimped valve assembly 28 will thus require destruction of the discharge actuator 117. FIGS. 11A-11G illustrate a cap member 120 suitable for use with the discharge actuator 116, 117. The cap member 120 includes a depending lip 128 which depends from the cover plate 130. The depending lip 128 includes a snap flange 132 constructed and arranged to snap over the outer diameter of the discharge actuator 116, 117 to hold the cap 120 in position to prevent inadvertent actuation of the discharge actuator 116, 117. The cap 120 is removable and replaceable to the discharge actuator 116, 117.

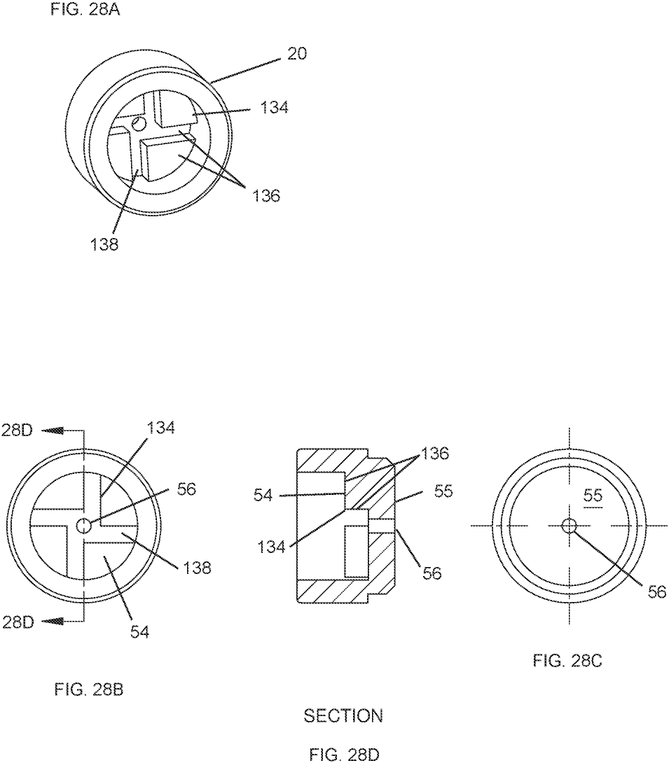

Referring generally to the Figures, and more specifically to FIGS. 28A-30F, various embodiments of breakup nozzles 20 are illustrated. The breakup nozzle 20 includes a rear breakup surface 54, and a front surface 55 including the nozzle orifice 56. The rear breakup surface 54 includes at least one shearing corner 134, and more preferably a plurality of shearing corners 134. In general, the shearing corners 134 are sharp corners constructed from two adjoining planar surfaces 136, one of the planar surfaces 136 aligned parallel with respect to the longitudinal centerline of the core tube 18, and one of the planar surfaces 136 arranged perpendicular to the core tube 18. In this manner, the liquefied endothermic gas directed at the rear breakup surface 54 impinges the rear surface 54 adjacent the nozzle orifice 56, wherein the liquid breaks up and expands to exit the orifice 56 as a vapor with velocity. In a most preferred embodiment, the at least one shearing corner 134 is a sharp corner constructed from three adjoining planar surfaces 136, constructing a U-shaped breaker channel 138, two of the planar surfaces 136 aligned parallel with respect to the longitudinal centerline of the core tube 18, and one of the planar surfaces 136 arranged perpendicular to the longitudinal centerline of the core tube 18. In another embodiment, the rear surface 54 of the breakup nozzle 20 includes four of the U-shaped breaker channels 138. The breaker channels 138 may be arranged at right angles with respect to each other, breaking the rear surface into four quadrants. The core tube 18 is constructed and arranged to direct the liquefied endothermic gas at the rear breakup surface 54 at an acute angle with respect to the longitudinal centerline of the core tube 18 to break the liquefied endothermic gas into a vapor which is directed at a skin surface to decrease the temperature of the skin suitably to cause numbness of the skin. Because of its physical properties, the heat-absorbing endothermic vapor constricts blood flow at the injection site, and temporary numbing occurs. The endothermic vapor stops the propagation of the painful nerve stimuli, and the patient feels the tactile, or pressure, as opposed to the pain sensation. According to the Gate theory, the pressure nerve fibers supersede the painful nerve fibers so that the mechanical contraction of the muscles blocks the transmission of pain perception. The use of the endothermic vapor also temporarily distracts the patient by creating a noise that diverts the patient’s attention away from any potential or anticipated pain. Finally, because the endothermic vapor blanches the mucosa, a readily-visible target is created for insertion of the needle to assure that the deadened area is not bypassed. When the practitioner observes that the injection site mucosa has been blanched, the site is effectively deadened and a painless, concomitant injection is possible. The practitioner can then quickly inject the injection site by inserting the injection needle 38 and depressing the plunger 34. Because of the positioning of the vapor outlet nozzle 20 with respect to the needle 38, the dispersal of the vapor and subsequent injection of anesthetic can be accomplished almost concurrently, and with no pain to the patient.

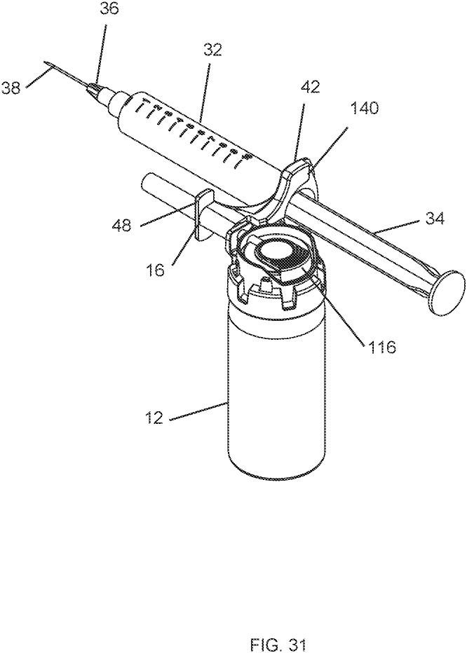

Referring generally to the Figures, and more specifically to FIGS. 31-37, an embodiment of the present device that includes a slide-in syringe retainer 140 is illustrated. The slide-in syringe retainer 140 includes a front wall 142 and a rear wall 144 having a gap therebetween to receive the opposing grips 39 of a syringe 14. The front wall 142 and rear wall 144 are formed or secured together and divided in a central portion to form two spring fingers 146. The opposing grips 39 are slid between the front wall 142 and the rear wall 144. The diameter of the syringe barrel 32 causes the spring fingers 146 to move slightly apart to snap over the syringe barrel 32. The spring pressure created by the spring fingers 146 retains the syringe barrel snugly to the cold spray module housing 16, while the opposing grips 39 positioned between the front and rear walls 142, 144 prevent rotation of the syringe 14 perpendicular to the longitudinal axis of the syringe during operation of the syringe 14.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. An endothermic vapor applicator attachable to a syringe comprising:
a cold spray module configured to be removably attached to the barrel portion of a syringe, said cold spray module including a body, said body including a core tube for directing a an endothermic liquid stream to impinge against a breaker surface for breaking the liquid stream into an endothermic vapor before the endothermic vapor is directed through a nozzle, the nozzle constructed and arranged to cause the endothermic vapor to be directed as a stream to intersect with a delivery axis of a needle connected to the syringe when the syringe is attached to the cold spray module;
a canister containing a liquefied endothermic gas, the canister fluidly connected through a connection to the cold spray module so that the liquefied endothermic gas is in fluid communication with the core tube.

2. The endothermic vapor applicator according to claim 1 wherein the cold spray module includes a syringe barrel securing assembly, the syringe barrel securing assembly including at least two members configured for securing said syringe barrel to the cold spray module.

3. The endothermic vapor applicator according to claim 2 wherein the at least two members configured for securing said syringe barrel in place includes two opposing spring loaded clamping members.

4. The endothermic vapor applicator according to claim 2 wherein the syringe barrel securing device includes a housing having a first upper portion and a second lower portion.

5. The endothermic vapor applicator according to claim 4 wherein the first upper portion comprises channels or slots sized and shaped to hold and allow said two opposing spring loaded clamping members to slide therein.

6. The endothermic vapor applicator according to claim 1 wherein the canister is a metal canister having a valve assembly crimped and sealed within an open end of said canister to create a sealed pressure canister, said valve assembly including a dispenser tube extending outwardly therefrom for connection to the cold spray module.

7. The endothermic vapor applicator according to claim 6 wherein the valve is finger operable to release the liquefied endothermic gas.

8. The endothermic vapor applicator according to claim 1 wherein the breaker surface is a rear surface of the nozzle, the liquefied endothermic gas directed at the rear surface to impinge the rear surface adjacent a nozzle orifice, wherein the vaporized breaks up and expands to exit the orifice with velocity.

9. The endothermic vapor applicator according to claim 8 wherein the rear surface of the nozzle includes at least one shearing corner.

10. The endothermic vapor applicator according to claim 9 wherein the rear surface of the nozzle includes a plurality of shearing corners.

11. The endothermic vapor applicator according to claim 9 wherein the at least one shearing corner is a sharp corner constructed from two adjoining planar surfaces, one of the planar surfaces aligned parallel with respect to the longitudinal centerline of the core tube, and one of the planar surfaces arranged perpendicular to the core tube.

12. The endothermic vapor applicator according to claim 9 wherein the at least one shearing corner is a pair of sharp corners constructed from three adjoining planar surfaces, constructing a U-shaped channel, two of the planar surfaces aligned parallel with respect to the longitudinal centerline of the core tube, and one of the planar surfaces arranged perpendicular to the core tube.

13. The endothermic vapor applicator according to claim 12 wherein the rear surface of the nozzle includes four of the U-shaped channels.

14. The endothermic vapor applicator according to claim 13 wherein the four U-shaped channels are arranged at right angles with respect to each other, breaking the rear surface into four quadrants.

15. The endothermic vapor applicator according to claim 8 wherein the core tube is constructed and arranged to direct the liquefied endothermic gas at the rear breakup surface of the nozzle at an acute angle with respect to the longitudinal centerline of the core tube.

16. The endothermic vapor applicator according to claim 1 wherein said liquefied endothermic gas decreases the temperature of skin to cause numbness.
